(19) United States
(12) Patent Application Publication
(10) Pub. No.: US 2026/0306819 A1
(43) Pub. Date: Oct. 8, 2026

(54) PROTECTIVE AND GUIDING SYSTEM FOR HEMOSTASIS VALVE

(71) Applicant: Boston Scientific Scimed, Inc., Maple Grove, MN (US)

(72) Inventors: Foss Jiao, Vaughan (CA); Brian Ma, Etobicoke (CA); Mahban Samiee-Zafarghandy, Oakville (CA); Asher Lai, Markham (CA); Yan Yue, Mississauga (CA)

(21) Appl. No.: 19/637,919

(22) Filed: Apr. 2, 2026

Related U.S. Application Data
(60) Provisional application No. 63/782,465, filed on Apr. 2, 2025.

(51) Int. Cl. A61M 39/06; A61M 25/00; A61M 25/06; A61M 39/00

(52) U.S. Cl. CPC A61M 39/06; A61M 25/0097; A61M 25/0662; A61M 2039/0009

(57) ABSTRACT

A protecting and guiding system for a hemostasis valve includes a hub having a distal end and a proximal end opposite the distal end. A hollow chamber extends between the distal end and the proximal end of the hub. A hemostasis valve is positioned within the hollow chamber. The hemostasis valve is configured to receive a medical device and to prevent fluid from escaping from the hollow chamber. A guide plate is positioned proximal of the hemostasis valve. The guide plate is configured to guide the medical device towards a center of the hemostasis valve to protect the hemostasis valve from damage. A cap is configured to surround a portion of the hub to secure the hemostasis valve and the guide plate within the hollow chamber.

### CROSS REFERENCE TO RELATED APPLICATIONS

The present application claims the benefit of and priority to U.S. Provisional Patent Application No. 63/782,465, filed Apr. 2, 2025, the disclosure of which is incorporated herein in its entirety.

### TECHNICAL FIELD

The present disclosure relates to medical systems and methods for protecting the integrity of a hemostasis valve associated with a tubular medical device. More specifically, the disclosure relates to a protecting and guiding system that protects a hemostasis valve from damage or tearing as a result of off centered insertion of a medical device.

### BACKGROUND

During minimally invasive procedures in cardiac electrophysiology or interventional cardiology, a sheath device is commonly used to facilitate cardiac catheterization. A hemostasis valve system is typically located near the proximal end of the sheath to permit therapy and accessory device to pass through. The hemostasis valve prevents blood loss and air ingress into the patient during the procedure. Hemostasis valves are normally made with elastomer material, like silicone.

Sometimes, when inserting a therapy or accessory device, such as a guidewire, stylet, perforation device, dilator, catheter, or other elongate medical device, into the sheath through the hemostasis valve, the device may not always point at the center of the valve. When inserting the therapy or accessory off centered, some undesired scenarios can occur such as difficulty with insertion, device buckling or kinking, and damage or tearing of the sheath hemostasis valve. The present disclosure aims to provide a solution to off centered insertion of therapy or accessory devices into hemostasis valves.

### SUMMARY

Example 1 is protecting and guiding system for a hemostasis valve. The system includes a hub having a distal end and a proximal end opposite the distal end. A hollow chamber extends between the distal end and the proximal end of the hub. A hemostasis valve is positioned within the hollow chamber. The hemostasis valve is configured to receive a medical device and to prevent fluid from escaping from the hollow chamber. A guide plate is positioned proximal of the hemostasis valve. The guide plate is configured to guide the medical device towards a center of the hemostasis valve to protect the hemostasis valve from damage.

Example 2 is the system of Example 1, wherein the hollow chamber includes a narrow diameter portion and a wide diameter portion that is proximal of the narrow diameter portion, and an annular recess is formed in the wide diameter portion configured for receiving a portion of the hemostasis valve.

Example 3 is the system of Example 2, wherein the wide diameter portion includes an annular protrusion adjacent to the annular recess.

Example 4 is the system of Example 3, wherein the hemostasis valve includes a valve recess configured to receive the annular protrusion.

Example 5 is the system of any of Examples 1 to 4, further comprising a cap configured to surround a portion of the hub to secure the hemostasis valve and the guide plate within the hollow chamber.

Example 6 is the system of Example 5, wherein the cap includes at least one opening configured to receive a ramped protrusion of the hub.

Example 7 is the system of any of Examples 1 to 6, wherein the guide plate is in contact with the hemostasis valve within the hollow chamber.

Example 8 is the system of any of Examples 1 to 6, wherein the guide plate is spaced from the hemostasis valve within the hollow chamber.

Example 9 is the system of any of Examples 1 to 8, wherein the guide plate is configured as a cylindrical disc including at least one slit at a center thereof.

Example 10 is the system of Example 9, wherein the at least one slit includes a first slit orthogonal to a second slit.

Example 11 is the system of Example 9, wherein the at least one slit traverses a central opening of the guide plate.

Example 12 is the system of any of Examples 1 to 8, wherein the guide plate includes a conical distal surface and a funnel-shaped opening on the proximal surface, the funnel-shaped opening aligned with a center of the guide plate.

Example 13 is the system of any of Examples 1 to 12, further comprising a spacer including a distal side, a proximal side, and central opening, the spacer configured to space the hemostasis valve from the guide plate within the hollow chamber.

Example 14 is the system of Example 13, wherein the spacer includes a shoulder on the proximal side configured to hold the guide plate.

Example 15 is the system of Example 13, wherein the distal side of spacer includes an annular protrusion adjacent to an annular recess.

Example 16 is a protecting and guiding system for a hemostasis valve. The system includes a hub having a distal end and a proximal end opposite the distal end. A hollow chamber extends between the distal end and the proximal end of the hub. A hemostasis valve is positioned within the hollow chamber. The hemostasis valve is configured to receive a medical device and to prevent fluid from escaping from the hollow chamber. A guide plate is positioned proximal of the hemostasis valve. The guide plate is configured to guide the medical device towards a center of the hemostasis valve to protect the hemostasis valve from damage. A cap is configured to surround a portion of the hub to secure the hemostasis valve and the guide plate within the hollow chamber.

Example 17 is the system of Example 16, wherein the hollow chamber includes a narrow diameter portion and a wide diameter portion that is proximal of the narrow diameter portion, and an annular recess is formed in the wide diameter portion configured for receiving a portion of the hemostasis valve.

Example 18 is the system of Example 17, wherein the wide diameter portion includes an annular protrusion adjacent to the annular recess.

Example 19 is the system of Example 18, wherein the hemostasis valve includes a valve recess configured to receive the annular protrusion.

Example 20 is the system of Example 16, wherein the cap includes at least one opening configured to receive a ramped protrusion of the hub.

Example 21 is the system of Example 16, wherein the guide plate is in contact with the hemostasis valve within the hollow chamber.

Example 22 is the system of Example 16, wherein the guide plate is spaced from the hemostasis valve within the hollow chamber.

Example 23 is the system of Example 16, wherein the guide plate is configured as a cylindrical disc including at least one slit at a center thereof.

Example 24 is the system of Example 23, wherein the at least one slit includes a first slit orthogonal to a second slit.

Example 25 is the system of Example 23, wherein the at least one slit traverses a central opening of the guide plate.

Example 26 is the system of Example 16, wherein the guide plate includes a conical distal surface and a funnel-shaped opening on the proximal surface, the funnel-shaped opening aligned with a center of the guide plate.

Example 27 is the system of Example 16, further comprising a spacer including a distal side, a proximal side, and central opening, the spacer configured to space the hemostasis valve from the guide plate within the hollow chamber.

Example 28 is the system of Example 27, wherein the spacer includes a shoulder on the proximal side configured to hold the guide plate.

Example 29 is the system of Example 27, wherein the distal side of spacer includes an annular protrusion adjacent to an annular recess.

Example 30 is a protecting and guiding system for a hemostasis valve. The system includes a hub having a distal end and a proximal end opposite the distal end. A hollow chamber extends between the distal end and the proximal end of the hub. A hemostasis valve is positioned within the hollow chamber. The hemostasis valve is configured to receive a medical device and to prevent fluid from escaping from the hollow chamber. A guide plate is positioned proximal of the hemostasis valve. The guide plate is configured to guide the medical device towards a center of the hemostasis valve to protect the hemostasis valve from damage. A spacer is configured to space the hemostasis valve from the guide plate within the hollow chamber. A cap configured to surround a portion of the hub secures the hemostasis valve and the guide plate within the hollow chamber.

Example 31 is the system of Example 30, wherein the guide plate is configured as a cylindrical disc including at least one slit at a center thereof.

Example 32 is the system of Example 31, wherein the at least one slit traverses a central opening of the guide plate.

Example 33 is the system of Example 30, wherein the guide plate includes a conical distal surface and a funnel-shaped opening on the proximal surface, the funnel-shaped opening aligned with a center of the guide plate.

Example 34 is a sheath for performing a medical procedure. The sheath includes an elongate body having a distal end and a proximal end opposite the distal end. A hub is connected to the proximal end of the elongate body. The hub has a distal end and a proximal end opposite the distal end. A hollow chamber extends between the distal end and the proximal end of the hub. A hemostasis valve is positioned within the hollow chamber. The hemostasis valve is configured to receive a medical device and to prevent fluid from escaping from the hollow chamber. A guide plate is positioned proximal of the hemostasis valve. The guide plate is configured to guide the medical device towards a center of the hemostasis valve to protect the hemostasis valve from damage. A cap configured to surround a portion of the hub secures the hemostasis valve and the guide plate within the hollow chamber.

Example 35 is the sheath of Example 34, further comprising a spacer configured to space the hemostasis valve from the guide plate within the hollow chamber.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a sheath including a protecting and guiding system for a hemostasis valve, in accordance with embodiments of the disclosure.

FIG. 2A is a cross-sectional view of the protecting and guiding system of FIG. 1 illustrating a medical device being inserted into the protecting and guiding system off-centered, in accordance with embodiments of the disclosure.

FIG. 2B is a cross-sectional view of the protecting and guiding system of FIG. 1 illustrating a medical device being inserted into the protecting and guiding system along a central axis of the protecting and guiding system, in accordance with embodiments of the disclosure.

FIGS. 3A and 3B are perspective views of a guide plate in the form of a cylindrical disc, in accordance with embodiments of the disclosure.

FIG. 4 is a perspective view of a guide plate in the form of a conical disc, in accordance with embodiments of the disclosure.

FIG. 5 is a cross-sectional view of an alternate embodiment of the protecting and guiding system, in accordance with embodiments of the disclosure.

FIG. 6 is cross-sectional view of an alternate embodiment of the protecting and guiding system, in accordance with embodiments of the disclosure.

FIG. 7 is a perspective view of a valve plate, in accordance with embodiments of the disclosure.

FIG. 8A is a top view of an alternate embodiment of the valve plate, in accordance with embodiments of the disclosure.

FIG. 8B is a bottom perspective view of the valve plate, in accordance with embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

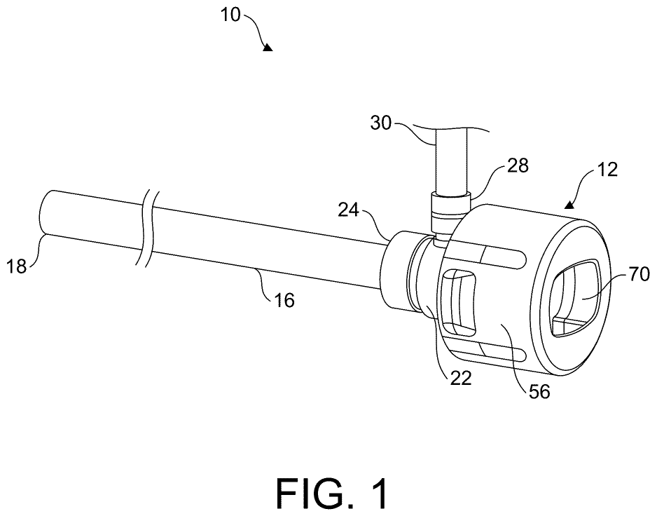

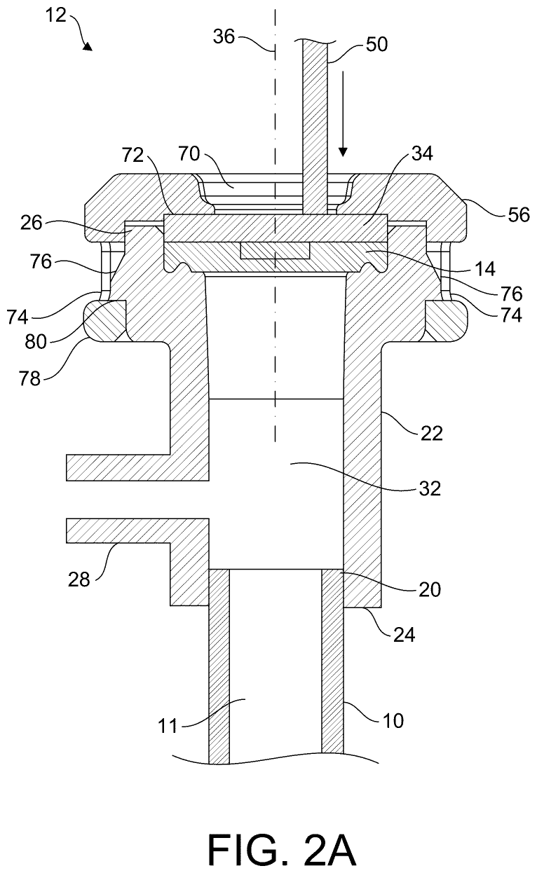

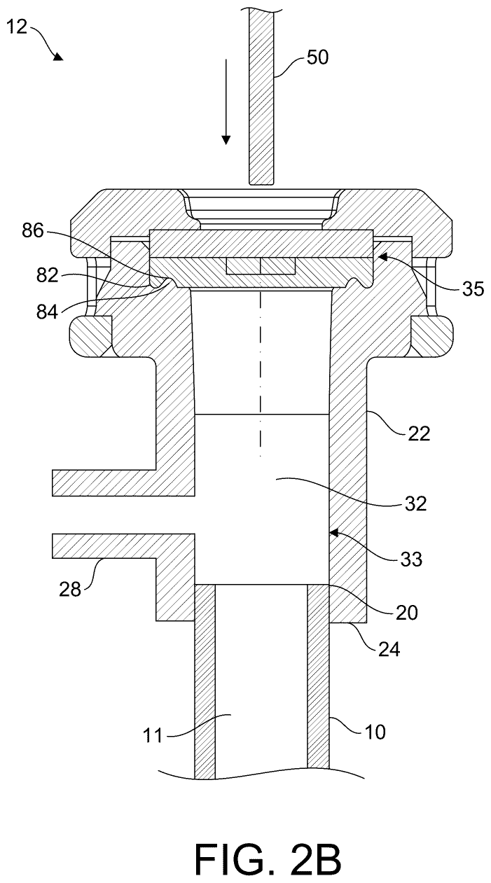

FIG. 1 is a perspective view of a sheath 10 including a protecting and guiding system 12 for a hemostasis valve 14 (shown in FIG. 2A), in accordance with embodiments of the disclosure. The sheath 10 of FIG. 1 is configured for performing a medical procedure, for example cardiac catheterization or a transseptal crossing procedure. The sheath 10 can include any catheter, guide catheter, guide sheath, dilator, or other tubular medical device. The sheath 10 includes an elongate body 16 having a distal end 18 and a proximal end 20 opposite the distal end 18. The elongate body 16 is hollow and includes a lumen 11 (shown in FIGS. 2A and 2B) extending from the distal end 18 to the proximal end 20. The protecting and guiding system 12 is configured to guide a medical device through the center of a hemostasis valve 14 in order to prevent the hemostasis valve 14 from tearing due to the medical device being inserted off center. During use, a medical device 50 is inserted through the protecting and guiding system 12 into the lumen 11 of the sheath 10. In some embodiments, the medical device 50 is translated along the lumen 11 for delivery out of the distal end 18 of the sheath 10.

The protecting and guiding system 12 includes a hub 22 connected to the proximal end 20 of the elongate body 16, and components within the hub 22 and attached to the hub 22. Some embodiments of the hub 22 include a side port 28 for connection to a conduit 30. The side port 28 can be located along any portion the hub 22. For example, the side port 28 is located closer to the sheath 10 in some embodiments, and further from the sheath 10 in other embodiments. The conduit 30 is configured to remove fluid from the lumen 11 of the sheath 10 or deliver fluid into the lumen 11 of the sheath 10. The hub 22 has a distal end 24 and a proximal end 26 (shown in FIGS. 2A and 2B) opposite the distal end 24.

As shown in FIGS. 2A and 2B, a hollow chamber 32 extends between the hub distal end 24 and the hub proximal end 26. The hollow chamber 32 is in fluid communication with the lumen 11 of the sheath 10. A hemostasis valve 14 is positioned within the hollow chamber 32. The hemostasis valve 14 prevents fluid from escaping from the hollow chamber 32. The hemostasis valve 14 is configured to receive a medical device 50 and to seal around the medical device 50. The hemostasis valve 14 is formed of an elastomeric material, such as silicone.

A guide plate 34 is positioned proximal of the hemostasis valve 14. As illustrated in FIGS. 2A and 2B, the guide plate 34 is in contact with the hemostasis valve 14. The guide plate 34 is configured to guide the medical device 50 towards a center of the hemostasis valve 14 to protect the hemostasis valve 14 from damage. As shown in FIG. 2A, the medical device 50 is being inserted into the hub 22 offset from the longitudinal axis 36 of the hub 22. The guide plate 34 guides the medical device 50 towards the central longitudinal axis 36 as shown in FIG. 2B so the medical device 50 can pass through the center of the hemostasis valve 14 without damaging the hemostasis valve 14. The guide plate 34 is formed from an elastomer or plastic material, such as silicone. In some embodiments, the guide plate 34 is formed of a material having a durometer higher than a material forming the hemostasis valve. In some embodiments, the guide plate 34 has a durometer in the range of Shore A 20-80. This allows for increased tear resistance of the guide plate 34. In some embodiments, the guide plate 34 is lubricated. For example, the guide plate 34 can be lubricated with silicone oil. The guide plate 34 is configured to deflect radially or towards the hemostasis valve 14 to allow for the medical device 50 to pass through.

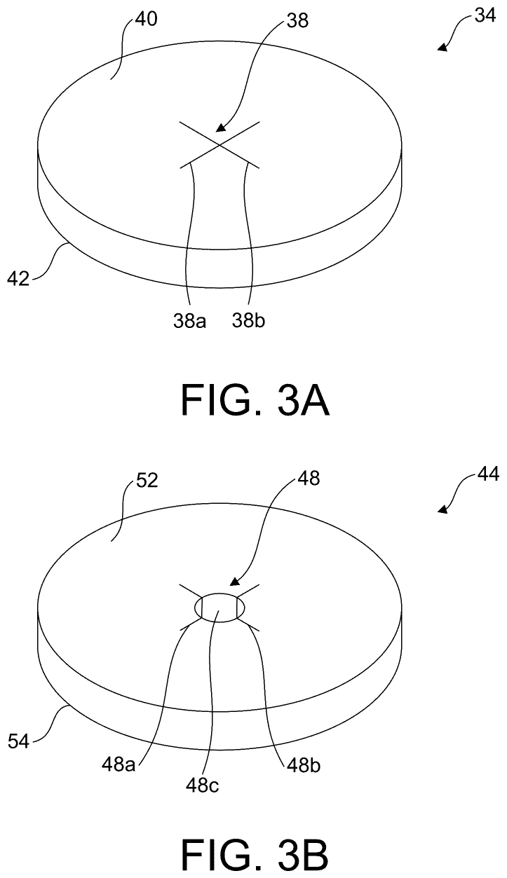

FIG. 3A illustrates a perspective view of a guide plate 34 in the form of a cylindrical disc, in accordance with embodiments of the disclosure. The guide plate 34 has a circular proximal surface 40, a circular distal surface 42, and a constant thickness between the circular proximal surface 40 and the circular distal surface 42. The guide plate 34 includes a centering feature 38 located at a center of the guide plate 34. The centering feature 38 in FIG. 3A takes the form of at least one slit 38a, 38b that extends between the circular proximal surface 40 and the circular distal surface 42. The at least one slit 38a, 38b includes a first slit 38a that is orthogonal to a second slit 38b. When a medical device 50 is inserted into the hub 22 off set from the longitudinal axis, the guide plate 34 prevents the medical device 50 from contacting the hemostasis valve 14 until the medical device 50 aligns with the centering feature 38. Once aligned with the centering feature 38, the medical device 50 passes through the guide plate 34 and through an opening of the hemostasis valve 14 into the hollow chamber 32. The centering feature 38 provides the guide plate 34 with a variable stiffness. The center of the guide plate 34 is less stiff due to the centering feature 38. Therefore, the center of the guide plate 34 deflects when the medical device 50 interacts with the guide plate 34. This deflection funnels the medical device towards the center and through the opening of the hemostasis valve 14.

FIG. 3B illustrates a perspective view of another embodiment of a cylindrical disc guide plate 44. The centering feature 48 in FIG. 3B takes the form of at least one slit 48a, 48b and a central opening 48c. The at least one slit 48a, 48b extends between a circular proximal surface 52 and a circular distal surface 54. The at least one slit 48a, 48b includes a first slit 48a that is orthogonal to a second slit 48b. The first slit 48a and the second slit 48b traverse the central opening 48c. When a medical device 50 is inserted into the hub 22 off set from the longitudinal axis, the guide plate 44 prevents the medical device 50 from contacting the hemostasis valve 14 until the medical device 50 aligns with the centering feature 48. Once aligned with the centering feature 48, the medical device 50 passes through the guide plate 44 and through an opening of the hemostasis valve 14 into the hollow chamber 32. In other embodiments, the centering feature 38 takes the form of a spiral or helical slit. The centering feature 48 provides the guide plate 44 with a variable stiffness. The center of the guide plate 44 is less stiff due to the centering feature 48. Therefore, the center of the guide plate 44 deflects when the medical device 50 interacts with the guide plate 44. This deflection funnels the medical device towards the center and through the opening of the hemostasis valve 14.

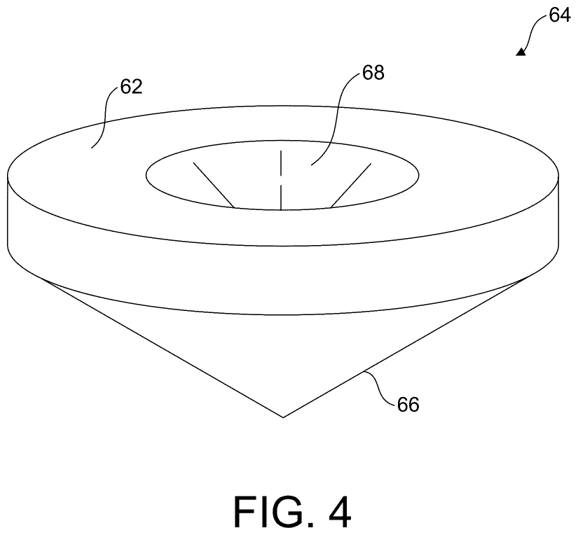

FIG. 4 is a perspective view of a guide plate 64 in the form of a conical disc, in accordance with embodiments of the disclosure. The guide plate 64 includes a proximal surface 62 and a conical distal surface 66. The proximal surface includes an opening 68 leading from the proximal surface 62 to the conical distal surface 66. The opening 68 is funnel-shaped having a larger diameter adjacent the proximal surface 62 and a smaller diameter adjacent the conical distal surface 66. The funnel-shaped opening 68 is aligned with a center of the guide plate 64 such that a medical device 50 is guided to the opening of the hemostasis valve.

Referring again to FIGS. 1, 2A, and 2B, a cap 56 is configured to surround a proximal portion of the hub 22. The cap 56 secures the hemostasis valve 14 and the guide plate 34 within the hollow chamber 32. The cap 56 includes an opening 70 for introduction of the medical device 50 therein. The cap 56 includes an inner surface 72 that faces and/or contacts the guide plate 34. In some embodiments, the cap 56 is secured to the hub 22 by adhesives, welding, or other means for permanently attaching the cap 56 to the hub 22. In other embodiments, the cap 56 is removably secured to the hub 22 by a snap-fit mechanism or threaded connection. As illustrated in FIGS. 1, 2A, and 2B, the cap 56 includes at least one opening 74 configured to receive a ramped protrusion 76 of the hub 22. When placing the cap 56 on the hub 22, a lip 78 adjacent the at least one opening 74 flexes outwardly over the ramped protrusion 76 until the at least one opening 74 aligns with the ramped protrusion 76 and receives the ramped protrusion 76 therein. The lip 78 then contracts to an original configuration and abuts a distal protrusion surface 80 to securely hold the cap 56 in place.

As illustrated in FIGS. 2A and 2B, the hollow chamber 32 includes a narrow diameter portion 33 and a wide diameter portion 35 that is proximal of the narrow diameter portion 33. An annular recess 82 is formed in the wide diameter portion 35. The annular recess 82 is configured for receiving a portion of the hemostasis valve 14. An annular protrusion 84 is located adjacent to the annular recess 82. The hemostasis valve 14 is located in and held by the annular recess 82. In some embodiments, the hemostasis valve 14 includes a valve recess 86 that is configured to receive the annular protrusion 84. In other embodiments, the hemostasis valve 14 is pliable and is compressed by the annular protrusion 84.

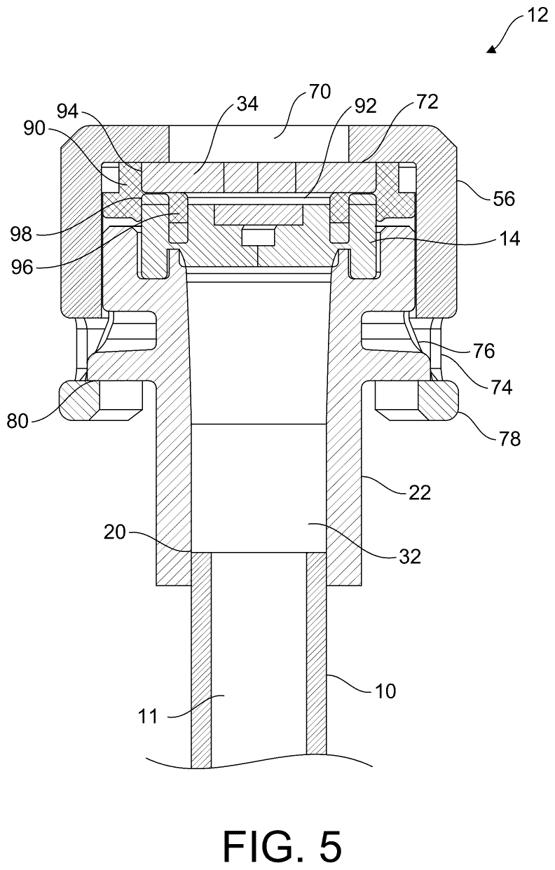

FIG. 5 is a cross-sectional view of an alternate embodiment of the protecting and guiding system 12, in accordance with embodiments of the disclosure. Like reference numerals are used to annotate similar structure where possible. The protecting and guiding system 12 illustrated in FIG. 5 includes a cap 56 configured to surround a proximal portion of the hub 22. The cap 56 secures a hemostasis valve 14, a guide plate 34, and a spacer 90 within the hollow chamber 32.

The spacer 90 is configured to space the hemostasis valve 14 from the guide plate 34 within the hollow chamber 32 of the hub 22. The spacer 90 includes a distal side facing the hemostasis valve 14 and a proximal side facing the guide plate 34. The spacer 90 is in contact with both the hemostasis valve 14 and the guide plate 34. A central opening 92 provides a gap between the hemostasis valve 14 and the guide plate 34 and allows for a medical device 50 to pass therethrough. The central opening 92 also serves as a buffer preventing the guide plate 34 from pressing against the hemostasis valve 14. In some situations, if the guide plate 34 contacts and presses against the hemostasis valve 14, the seal provided by the hemostasis valve 14 can potentially be broken. The central opening 92 spaces the guide plate 34 from the hemostasis valve 14 so the integrity of the hemostasis valve 14 can be maintained.

The proximal side of the spacer 90 includes a shoulder 94 configured to hold the guide plate 34. The distal side of spacer 90 includes an annular protrusion 96 adjacent to an annular recess 98 that are configured to mate with a corresponding recess and protrusion of the hemostasis valve 14. In some embodiments, the spacer 90 applies compression to the hemostasis valve 14.

The cap 56 includes an opening 70 for introduction of the medical device 50 therein. The cap 56 includes an inner surface 72 that contacts the guide plate 34. In some embodiments, the cap 56 is secured to the hub 22 by adhesives, welding, or other means for permanently attaching the cap 56 to the hub 22. In other embodiments, the cap 56 is removably secured to the hub 22 by a snap-fit mechanism or threaded connection. As illustrated in FIG. 5 the cap 56 includes at least one opening 74 configured to receive a ramped protrusion 76 of the hub 22. When placing the cap 56 on the hub 22, a lip 78 adjacent the at least one opening 74 flexes outwardly over the ramped protrusion 76 until the at least one opening 74 aligns with the ramped protrusion 76 and receives the ramped protrusion 76 therein. The lip 78 then contracts to an original configuration and abuts a distal protrusion surface 80 to securely hold the cap 56 in place. In some embodiments, the guide plate 34 functions as a secondary seal around the medical device 50.

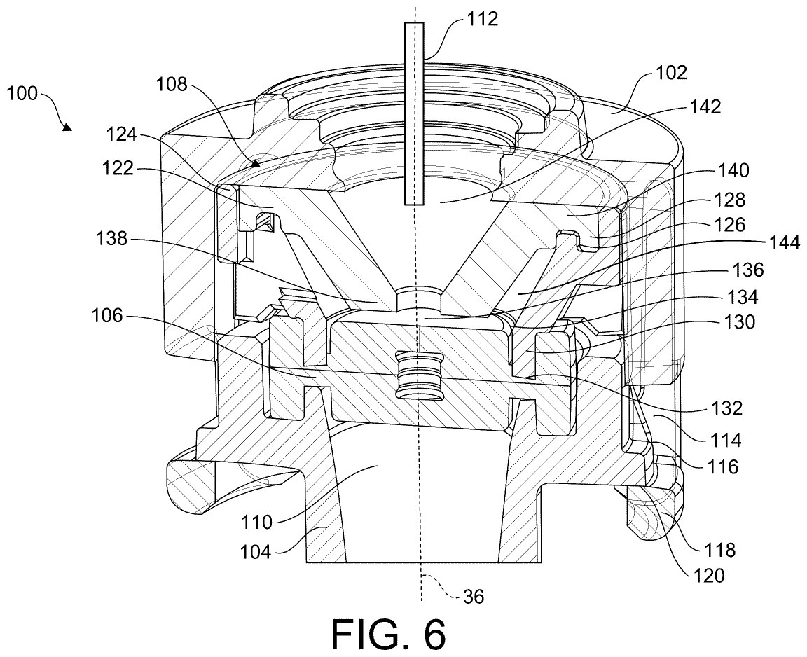

FIG. 6 is a cross-sectional view of an alternate embodiment protecting and guiding system 100. In the illustrated embodiment, the protecting and guiding system 100 includes a cap 102, a hub 104, a hemostasis valve 106, and a guide plate 108. The cap 102 is configured to surround a proximal portion of the hub 104. The cap 102 secures the hemostasis valve 106 and the guide plate 108 within the hollow chamber 110. The hemostasis valve 106 is configured to receive a medical device 112 and to seal around the medical device 112. In the illustrated embodiment, the hemostasis valve 106 is formed of an elastomeric material, such as silicone.

In the illustrated embodiment, the cap 102 is removably secured to the hub 104 by a snap-fit mechanism. In the illustrated embodiment, the cap 102 includes an opening 114 configured to receive a ramped protrusion 116 of the hub 104. When placing the cap 102 on the hub 104, a lip 118 adjacent the opening 114 flexes outwardly over the ramped protrusion 116 until the one opening 114 aligns with the ramped protrusion 116 and receives the ramped protrusion 116 therein. The lip 118 then contracts to an original configuration and abuts a distal protrusion surface 120 to securely hold the cap 102 in place.

In the illustrated embodiment, the guide plate 108 is positioned proximal of the hemostasis valve 106. In the illustrated embodiment, the guide plate 108 includes a guiding valve 122 and a valve plate 124. The valve plate 124 comprises a U-shaped annular recess 126 configured to receive an annular ridge 128 on the guiding valve 122, such that the guiding valve 122 fits into the valve plate 124. In some embodiments, the guiding valve 122 couples with (e.g., snaps into) the valve plate 124. The valve plate 124 provides structural support for the guiding valve 122.

In the illustrated embodiment, the guide plate 108 is configured to guide the medical device 112 towards the center of the hemostasis valve 106 (e.g., the central longitudinal axis 36) to protect the medical device 112 from damaging the hemostasis valve 106. The guiding valve 122 acts as a deflecting component to guide the medical device 112 to the central longitudinal axis 36. In the illustrated embodiment, the guide plate 108 is formed from an elastomer or plastic material, such as silicone. In some embodiments, the guide plate 108 is formed of a material having a durometer higher than a material forming the hemostasis valve 106. The guide plate 108 is configured to deflect radially and/or towards the hemostasis valve 106 to allow for the medical device to pass through.

In the illustrated embodiment, the valve plate 124 comprises a distal annular ridge 130 configured to fit into an annular recess 132 on the hemostasis valve 106. The valve plate 124 and the hemostasis valve 106 are positioned such that an annular gap 134 exists between the valve plate 124 and the hemostasis valve 106, thereby preventing the valve plate 124 from compressing onto the hemostasis valve 106. In the illustrated embodiment, the guiding valve 122 and the valve plate 124 are positioned relative to the hemostasis valve 106 such that an axial secondary gap 136 exists between the guiding valve 122 and the hemostasis valve 106. The secondary gap 136 ensures that the guiding valve 122 does not compress the hemostasis valve 106 or force the hemostasis valve 106 open. In some embodiments, the secondary gap 136 accommodates tolerance variation among the components to ensure that, across the full range of dimensional variation, the guiding valve 122 does not inadvertently press the hemostasis valve 106 open.

In the illustrated embodiment, the guiding valve 122 comprises a frustoconical geometry, such that a distal end 138 of the guiding valve 122 has a smaller diameter relative to a proximal end 140 of the guiding valve 122. The guiding valve 124 includes a bore 142 extending from the proximal end 140 to the distal end 138. The bore 140 is funnel-shaped, having a larger diameter at the proximal end 140 and a smaller diameter at the distal end 138. The proximal end 140 of the bore 142 is aligned with the center of the guiding plate 108 (e.g., the central longitudinal axis 36) such that the medical device 112 is guided to the opening of the hemostasis valve 106.

In the illustrated embodiment, the guiding valve 122 is positioned relative to the valve plate 124 such that a radial clearance 144 is formed between the guiding valve 122 and the valve plate 124. The radial clearance 144 provides space for radial expansion of the guiding valve 122 when the medical device 112 is inserted into the protecting and guiding system 100. Thereby, the insertion force required to advance the medical device 112 is substantially smaller than if there wasn't a radial clearance 144.

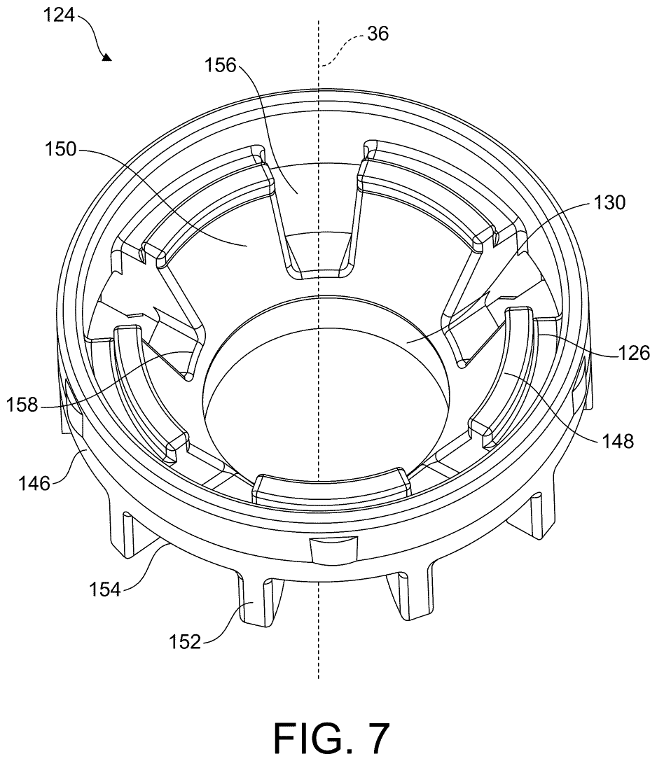

FIG. 7 is a perspective view of the valve plate 124. The valve plate 124 comprises a generally tapered cylindrical geometry including a proximal end surface 146 and the distal annular ridge 130.

In the illustrated embodiment, the valve plate 124 comprises a retention ring 148 located on a proximal end of an interior face 150. The retention ring 148 defines the annular recess 126 and holds the annular ridge 128 of the guiding valve 122 (shown in FIG. 6) in place after it couples with the valve plate 124. The retention ring 148 prevents the guiding valve 122 from being pulled toward the central longitudinal axis 36 as the medical device 112 is inserted into the protecting and guiding system 100 (shown in FIG. 6).

In the illustrated embodiment, the valve plate 124 comprises ten equally spaced and sized axial protrusions 152. In some embodiments, the valve plate 124 comprises greater or fewer axial protrusions 152 (e.g., three axial protrusions 152). The axial protrusions 152 extend distally from a distal surface 154 distal of the retention ring 148 and have a radial extent that matches that of a radially outer surface 146. Essentially, the axial protrusions 152 extend the radially outer surface 146 distally, which increases the exterior contact area with the cap 102 (shown in FIG. 6). Thereby, the axial protrusions 152 aid in aligning the valve plate 124 and the guiding valve 122 with the cap 102. Additionally, the axial protrusions 152 prevent the hemostasis valve 106 from being over-compressed as the cap 102 is removably secured to the hub 104 (shown in FIG. 6).

In the illustrated embodiment, the valve plate 124 comprises five equally spaced and sized vent holes 156. In some embodiments, the valve plate 124 comprises greater or fewer vent holes (e.g., at least ten vent holes 156). In the illustrated embodiment, the vent holes 156 are spaced circumferentially around the valve plate 124 and extend radially from the interior face 150 to the radially outer surface 146 between the distal surface 154 and a proximal surface 158 proximal to the distal annular ridge 130. The vent holes 156 facilitate the passage of air through the valve plate 124. Thereby the vent holes 156 prevent air from becoming trapped between the guiding valve 122 and the hemostasis valve 106, which could lead to the air being forced through the hemostasis valve 106 during insertion of the medical device 112 (shown in FIG. 6). The vent holes 156 remain unobstructed during radial deflection of the guiding valve 122 (e.g., during insertion of the medical device 112), so they maintain open passageways through the valve plate 124, thereby venting trapped air to the atmosphere.

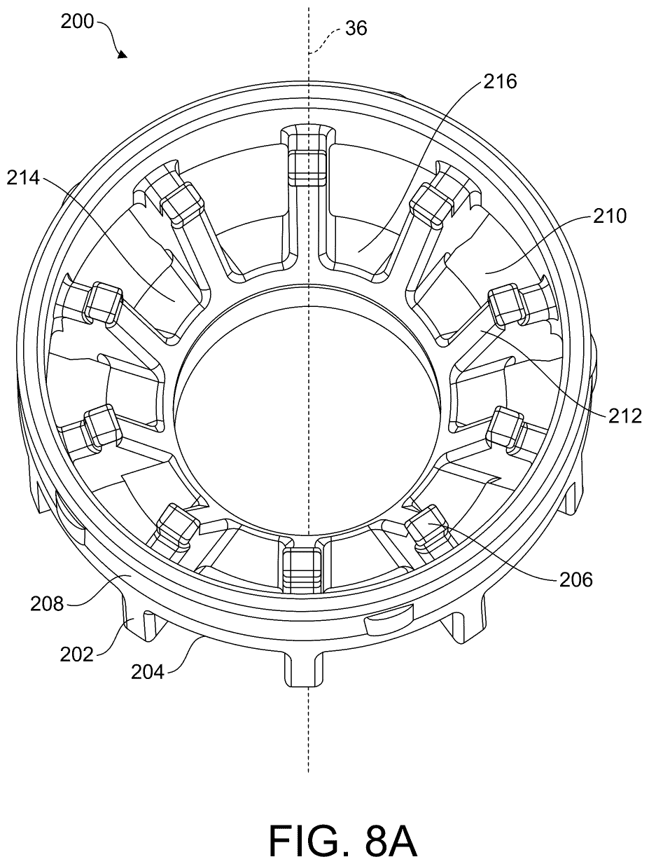

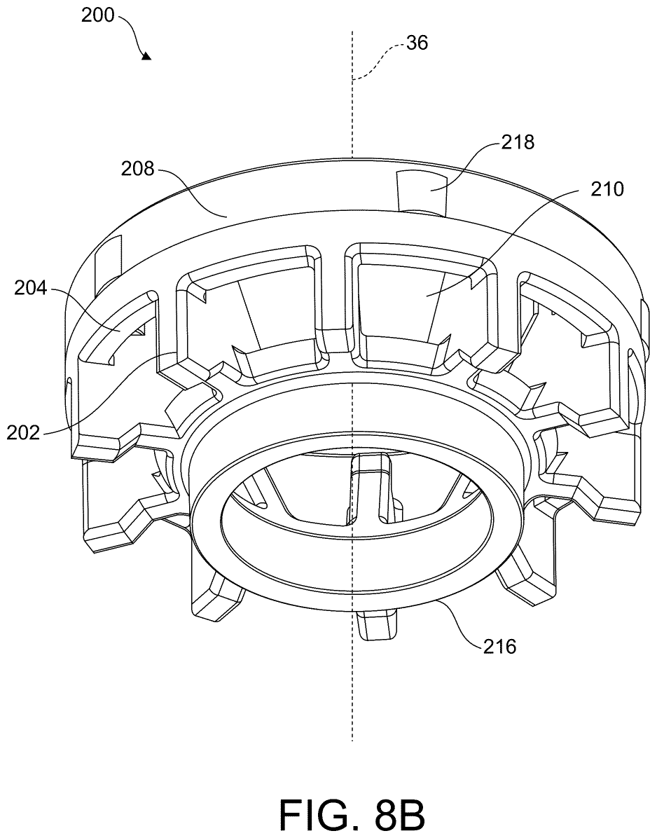

FIG. 8A is a top perspective view of an alternate embodiment of the valve plate 200. FIGS. 8A-8B will be discussed in conjunction with one another. The valve plate 200 is similar to the valve plate 144 with some differences, for example the axial protrusions 202 and the vent holes 204.

In the illustrated embodiment, the valve plate 200 comprises ten equally spaced and sized axial protrusions 202. In some embodiments, the valve plate 200 comprises greater or fewer axial protrusions 202. The axial protrusions 202 extend distally from a distal surface 204 of a retention ring 206 and have an outward radial extent that matches that of a radially outer surface 208 of the retention ring 206. Essentially, the axial protrusions 202 extend the radially outer surface 208 distally, which increases the exterior contact area with the cap 102 (shown in FIG. 6). Thereby, the axial protrusions 202 aid in aligning the valve plate 200 and the guiding valve 122 with the cap 102. Additionally, the axial protrusions 202 prevent the hemostasis valve 106 from being over-compressed as the cap 102 is removably secured to the hub 104 (shown in FIG. 6).

In the illustrated embodiment, the valve plate 200 comprises ten equally spaced and sized vent holes 210. In some embodiments, the valve plate 200 comprises greater or fewer vent holes 210. In the illustrated embodiment, the vent holes 210 are spaced circumferentially around the valve plate 200 and extend radially from the interior face 212 to the radially outer surface 208 between the distal surface 204 and a proximal surface 214 of a distal annular ring 216. The vent holes 210 facilitate the passage of air through the valve plate 200. Thereby, the vent holes 210 prevent air from becoming trapped between the guiding valve 122 and the hemostasis valve 106, which could lead to the air being forced through the hemostasis valve 106 during insertion of the medical device 112 (shown in FIG. 6). The vent holes 210 remain unobstructed during radial deflection of the guiding valve 122 (e.g., during insertion of the medical device 112), so they maintain open passageways through the valve plate 124, thereby venting trapped air to the atmosphere.

FIG. 8B is a bottom perspective view of the valve plate 200. In the illustrated embodiment, each vent hole 210 is positioned circumferentially between a pair of axial protrusions 202, such that every vent hole 210 is defined by two axial protrusions 202.

In the illustrated embodiment, the radially outer surface 208 comprises a lock protrusion 218. The lock protrusion 218 is configured to interface with the cap 102 (shown in FIG. 6) to secure the guide plate 108 to the cap 102, thereby holding the guide plate 108 and the cap 102 together and preventing relative movement (e.g., spinning, rattling etc.) therebetween. In some embodiments, the lock protrusions 218 are included on the valve plate 124 (shown in FIG. 7).

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A protecting and guiding system for a hemostasis valve, the system comprising:
a hub having a distal end and a proximal end opposite the distal end, a hollow chamber extending between the distal end and the proximal end;
a hemostasis valve positioned within the hollow chamber, the hemostasis valve configured to receive a medical device and to prevent fluid from escaping from the hollow chamber;
a guide plate positioned proximal of the hemostasis valve, the guide plate configured to guide the medical device towards a center of the hemostasis valve to protect the hemostasis valve from damage; and
a cap configured to surround a portion of the hub to secure the hemostasis valve and the guide plate within the hollow chamber.

2. The system of claim 1, wherein the hollow chamber includes a narrow diameter portion and a wide diameter portion that is proximal of the narrow diameter portion, and an annular recess is formed in the wide diameter portion configured for receiving a portion of the hemostasis valve.

3. The system of claim 2, wherein the wide diameter portion includes an annular protrusion adjacent to the annular recess.

4. The system of claim 3, wherein the hemostasis valve includes a valve recess configured to receive the annular protrusion.

5. The system of claim 1, wherein the cap includes at least one opening configured to receive a ramped protrusion of the hub.

6. The system of claim 1, wherein the guide plate is in contact with the hemostasis valve within the hollow chamber.

7. The system of claim 1, wherein the guide plate is spaced from the hemostasis valve within the hollow chamber.

8. The system of claim 1, wherein the guide plate is configured as a cylindrical disc including at least one slit at a center thereof.

9. The system of claim 8, wherein the at least one slit includes a first slit orthogonal to a second slit.

10. The system of claim 8, wherein the at least one slit traverses a central opening of the guide plate.

11. The system of claim 1, wherein the guide plate includes a conical distal surface and a proximal surface with a funnel-shaped opening, the funnel-shaped opening aligned with a center of the guide plate.

12. The system of claim 1, further comprising a spacer including a distal side, a proximal side, and a central opening, the spacer configured to space the hemostasis valve from the guide plate within the hollow chamber.

13. The system of claim 12, wherein the distal side of spacer includes an annular protrusion adjacent to an annular recess.

14. The system of claim 1, wherein the guide plate comprises a guiding valve and a valve plate, and the guiding valve has a frustoconical shape.

15. A protecting and guiding system for a hemostasis valve, the system comprising:
a hub having a distal end and a proximal end opposite the distal end, a hollow chamber extending between the distal end and the proximal end;
a hemostasis valve positioned within the hollow chamber, the hemostasis valve configured to receive a medical device and to prevent fluid from escaping from the hollow chamber;
a guide plate positioned proximal of the hemostasis valve, the guide plate configured to guide the medical device towards a center of the hemostasis valve to protect the hemostasis valve from damage;
a spacer configured to space the hemostasis valve from the guide plate within the hollow chamber; and
a cap configured to surround a portion of the hub to secure the hemostasis valve and the guide plate within the hollow chamber.

16. The system of claim 15, wherein the guide plate is configured as a cylindrical disc including at least one slit at a center thereof.

17. The system of claim 16, wherein the at least one slit traverses a central opening of the guide plate.

18. The system of claim 15, wherein the guide plate includes a conical distal surface and a proximal surface with a funnel-shaped opening, the funnel-shaped opening aligned with a center of the guide plate.

19. A sheath for performing a medical procedure, the sheath comprising:
an elongate body having a distal end and a proximal end opposite the distal end;
a hub connected to the proximal end of the elongate body, the hub having a distal end and a proximal end opposite the distal end, a hollow chamber extending between the distal end and the proximal end;
a hemostasis valve positioned within the hollow chamber, the hemostasis valve configured to receive a medical device and to prevent fluid from escaping from the hollow chamber;
a guide plate positioned proximal of the hemostasis valve, the guide plate configured to guide the medical device towards a center of the hemostasis valve to protect the hemostasis valve from damage; and
a cap configured to surround a portion of the hub to secure the hemostasis valve and the guide plate within the hollow chamber.

20. The sheath of claim 19, further comprising a spacer configured to space the hemostasis valve from the guide plate within the hollow chamber.
